(19) United States
(12) Patent Application Publication
(10) Pub. No.: US 2025/0364137 A1
(43) Pub. Date: Nov. 27, 2025

(54) A COMPUTER SYSTEM FOR DIAGNOSTIC ASSESSMENTS AND A METHOD THEREOF

(71) Applicant: EVEDA IP PTY LTD, Fitzroy, Victoria (AU)

(72) Inventor: Zahid BAALA, Victoria (AU)

(73) Assignee: EVEDA IP PTY LTD, Fitzroy, Victoria (AU)

(21) Appl. No.: 18/867,085

(22) PCT Filed: Mar. 15, 2023
(86) PCT No.: PCT/AU2023/050180

§ 371 Date: Nov. 19, 2024

(30) Foreign Application Priority Data
Mar. 15, 2022 (AU) 2022900626

(51) Int. Cl. G16H 50/20; G16H 10/60; G16H 40/63; G16H 50/30

(52) U.S. Cl. CPC G16H 50/20; G16H 10/60; G16H 40/63; G16H 50/30

(57) ABSTRACT

A portable medical device for diagnostic assessments, wherein the portable medical device comprises: a plurality of medical instruments each medical instrument including one or more sensors, wherein each sensor of the one or more medical instruments is configured to receive one or more samples from body of a person having information of that body; a processor to determine a plurality of parameters based on the information from the one or more samples; a memory unit operably connected to the processor and configured to store machine readable instructions, the processor obtaining the machine-readable instructions from the memory unit, and being configured by the machine-readable instructions to compare the determined the plurality of parameters with predefined parameters to thereby determine health related information of the human body based on the comparison.

### TECHNICAL FIELD

The present invention generally relates to health technologies and in particular to a portable medical device for diagnostic assessments and a method thereof, to enable patients, doctors, hospitals and other health care organisation to keep posted with heath assessments.

### BACKGROUND

Health and healthcare is increasingly important and especially in regional areas. Populations in regional areas do not receive the attention and priority they deserve as hospitals and other health care facilities are prioritised in capital cities. Urbanisation and the influx of larger populations to urban areas as well as work culture has become a major cause of this change, and which results in increasing day to day medical emergencies and dealing with patients with chronic health conditions. The medical facilities are irregularly distributed and are not able to handle a lot of chronic patients at once. The availability and approachability of medical facilities, due to lack of infrastructure, has always been an issue. In the case of a surge of any kind of disease, the medical infrastructure starts to fail rapidly. However, with constant vigilance and more frequent monitoring of the patients, this problem can be mitigated by managing the patients at pre stages of disease and handling their medical issues in accordance with early detected stages of the patient.

The available systems in certain areas, including regional or developing countries may be outdated and fails to help people with chronic diseases, their families, and caregivers. Further, the existing systems fail to improve medical supply and to reduce healthcare costs. The medical devices and computer systems available are unable to assist in refining processes, reduce costs and increase revenues for care facilities and doctors' offices. In addition, the available systems lack in enabling suppliers of meters of vital functions to go mobile. On a broader aspect, the available systems fail due to gap in rural area monitoring which for instance include lack of infrastructure, lack of resources, high cost, lack of quality, lack of awareness. Further, the available systems fail due to gap in urban monitoring caused due to lack of time, lack of care support at home, increased burden of disease, need for comprehensive management, on-site/home expectations, and high cost.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others.

Any discussion of the background art throughout the specification should in no way be considered as an admission that such background art is prior art nor that such background art is widely known or forms part of the common general knowledge in the field in Australia or worldwide.

### SUMMARY

According to a first aspect of the invention a computer system is provided for diagnostic assessments, wherein the computer system comprising one or more medical instruments, wherein any sensor of the one or more medical instruments configured to receive one or more samples from body of a person having information of that body, determine one or more parameter based on the information from the one or more samples, a memory unit configured to store machine-readable instructions, and a processor operably connected with the memory unit, the processor obtaining the machine-readable instructions from the memory unit, and being configured by the machine-readable instructions to compare the determined one or more parameters with predefined parameters, determine a health related information of the human body based on the comparison, and determine a predictive analysis risk predictions, comorbidity alerts, decision support for doctors, corporates and government, based on the health related information; and a communication network configured to send the health related information to one or more user devices held by the person, health organisations and other stake holders.

The one or more medical instruments may be selected from a group comprising spirometer, electrocardiography sensor, glucose ketone analyser, electronic blood pressure (BP) monitor, Lipid Profiler, creatinine monitor, HbA1C monitor, Hb monitor, urine analyser, cardiovascular analyser, thyroid, renal, liver function analyser, haemoglobin and white blood cell (WBC) analyser, cancer screening, communicable disease detector, cancer identifier, portable X-Ray, uric acid/UREA profiler, Thermometer, stethoscope, pulse oximeter, CBP analyser, mammogram.

The one or more samples are selected from a group comprising blood, tissue, saliva, urine, faeces or a combination thereof.

The computer system may further comprise a plurality of wheels and back strap for quick and easy mobility.

The health related information is selected from a group comprising basic biometric data (including temperature), complete urine analysis, haemoglobin and white blood cell count, detailed cardiovascular function (electrocardiogram, heart rate), blood vitamin analysis, detailed diabetes blood glucose levels, renal function test, thyroid test, hypertension, liver function test, pulmonary function test, prostate function test, Cancer Markers, Tumour Markers, Infection Markers and many more like covid-19 antibody testing IgM, electrolyte measurement test (captured manually), and infectious disease tests (e.g., Malaria, Chikungunya, Typhoid, H1N1 etc.).

The communication network is wireless and internet-enabled with an integrated 3G, 4G or 5G communications module or dongle which may be internal or externally installed.

The processor may be further configured to receive health information related to one or more patients for diagnostic assessments from doctors, hospitals or health organizations, allow any patient of the one or more patients to view, share and maintain health data, preserving health records digitally and chronologically, update information about any patient of the one or more patients thereby helping hospitals, medical stores and diagnostic centres, facilitate one or more information dialogs between patients, doctors, hospitals and all involved parties, perform self-risk assessment tests for stress, and also examine risk to develop certain chronic diseases, and monitor prognosis and diagnosis of patients in real-time.

The one or more user devices are selected from, but not limited to, a laptop, a desktop PC, an AR/VR headset, a smartphone, or a tablet.

According to a second aspect of the invention, there is provided a computer implemented method for diagnostic assessments. The computer implemented method being implemented on a device that includes a processor executing machine-readable instructions obtained from a memory unit, corresponding to an application which, when executed, performs the computer-implemented method. The computer-implemented method comprises step of receiving one or more samples from body of a person having information of that body, determining one or more parameter based on the information from the one or more samples, comparing the determined one or more parameters with predefined parameters, determining a health related information of the human body based on the comparison, and determining a predictive analysis risk predictions, comorbidity alerts, decision support for doctors, corporates and government, based on the health related information, and sending the health related information to the person, health organisations and other stake holders.

The computer implemented method further comprises a step of receiving health information related to one or more patients for diagnostic assessments from doctors, hospitals or health organizations, allowing any patient of the one or more patients to view, share and maintain health data, preserving health records digitally and chronologically, updating information about any patient of the one or more patients thereby helping hospitals, medical stores and diagnostic centres, facilitating one or more information dialogs between patients, doctors, hospitals and all involved parties, performing self-risk assessment tests for stress, and also examine risk to develop certain chronic diseases, and monitoring prognosis and diagnosis of patients in real-time.

According to another aspect of the invention there is provided a portable medical device for diagnostic assessments, wherein the portable medical device comprises: one or more medical instruments each medical instrument including one or more sensors, wherein any sensor of the one or more medical instruments is configured to receive one or more samples from body of a person having information of that body; a processor to determine one or more parameters based on the information from the one or more samples; a memory unit operably connected to the processor and configured to store machine-readable instructions, the processor obtaining the machine-readable instructions from the memory unit, and being configured by the machine-readable instructions to compare the determined one or more parameters with predefined parameters, determine health related information of the human body based on the comparison, and determine one or more of: a predictive analysis risk predictions; comorbidity alerts; information for healthcare professionals, based on the health related information; and a communication network configured to send the health related information to one or more user devices.

According to another aspect of the invention there is provided a portable medical device for diagnostic assessments, wherein the portable medical device comprises: a plurality of medical instruments each medical instrument including one or more sensors, wherein each sensor of the one or more medical instruments is configured to receive one or more samples from body of a person having information of that body; a processor to determine a plurality of parameters based on the information from the one or more samples; a memory unit operably connected to the processor and configured to store machine-readable instructions, the processor obtaining the machine-readable instructions from the memory unit, and being configured by the machine-readable instructions to compare the determined the plurality of parameters with predefined parameters to thereby determine health related information of the human body based on the comparison.

Preferably, the determined health related information of the human body comprises one or more of: a predictive analysis risk predictions in some instances using big data and artificial intelligence; comorbidity alerts; and information for healthcare professionals, based on the health related information.

Preferably, the portable medical device for diagnostic assessments includes a communication network configured to send the health related information to one or more user devices.

Preferably, the portable medical device includes the one or more samples from body of a person having information of that body comprise one or more of the following: blood; tissue; saliva; urine; or faeces.

Preferably, the portable medical device includes the user devices comprise one or more of the following: a laptop computer; a desktop computer; a portable electronic device such as a mobile phone or tablet; a patient database.

Preferably, the processor determines the health related information of the human body is within about 15 minutes.

Preferably, the portable medical device comprises an integrated touchscreen control panel to control the operation of the device. In some embodiments, the portable medical device comprises WiFi and/or Bluetooth communications modules to communicate with one or more local portable electronic devices that enable patient registration and information to be uploaded to the portable medical device and/or cloud database storage.

Preferably, the portable medical device includes the predictive analysis risk predictions include predictions of one: diabetes; health comorbidities; cardiovascular disorders; stress related illness; kidney disease; respiratory disorders; liver disease; cancer. The predictive analysis utilised previous patient history, big data and/or artificial intelligence.

Preferably, the portable medical device includes the plurality of medical instruments includes one or more of the following integrated components: Pulmonary Function/Spirometer; ECG; Glucose Ketone Analyzer; Electronic BP Monitor; Lipid Profiler; Creatinine Monitor; HbA1C Monitor; Hb Monitor; Urine Analyser; Cardiovascular Monitor; Thyroid, Renal, Liver Function monitor; Hemoglobin and WBC; Immuno Anaylser; Uric Acid/UREA Profiler.

Preferably, the plurality of medical instruments includes one or more of the following external components: Cancer Detection; Communicable Disease Detection; Cancer Identifier; Portable X-Ray; Thermometer; Stethoscope; Pulse Oxymeter; CBP Analyser.

In some embodiments, the medical instruments include one or more of a chemistry analyser, urine analyser, disc microflow, spirometer and blood pressure monitor.

In some embodiments the medical instruments comprises a chemistry analyser adapted to receive lithium-heparinized whole blood, plasma, or serum samples collected by venous or fingertip blood. The chemistry analyser is used with a reagent disc into which the sample is placed. The reagent disc is then placed into the chemistry analyser which can then perform a plurality of tests using the sample and reagent disc simultaneously.

In some embodiments the chemistry analyser is adapted to determine one, a plurality of or all the following: (TP) Total Protein; (ALB) Albumin; (GLO) Globulin; (A/G) A/G; (ALT) Alanine aminotransferase; (AST) Aspartate aminotransferase; (GGT) γ-glutamyl transpeptidase; (ALP) Alkaline phosphatase; (TBIL) Total bilirubin; (DBIL) Direct Bilirubin; (IBIL) Indirect Bilirubin; (UA) Uric acid; (CRE) Creatinine; (UREA) Urea; (CK) Creatine Kinase; (CK-MB) Creatine kinase isoenzymes; (LDH) Lactate dehydrogenase; (a-TBDH) a-TBDH; (AMY) Amylase; (GLU Glucose); (GSP) Glucose Serum albumin; (HCY) Homocysteine; (TG) Triglyceride; (CHOL) Total cholesterol; (HDL-C) High density liptein cholesterol; (LDL-C) Low density liptein cholesterol; (K) Kalium; (Na) Nasodium; (CL) Chloride; (Ca2+) Calcium; (P) phosphorus; (Mg2+) Magnesium; (CO2) carbon dioxide; and (NH3) NH3.

In some embodiments the medical instruments comprises a fluorescence immunoassay analyzer that is matched with specific reagents adapted based on fluorescence immunochromatography for Fluorescence Immunoassay test of human samples. The fluorescence immunoassay analyzer is adapted to receive and test various human samples such as serum, plasma, whole blood, urine and saliva to assist diagnosis.

Preferably, the medical instruments includes a saliva and/or urine monitor for drug testing. Preferably the drug testing includes testing for amphetamine, methamphetamine, THC, cannabis (metabolite), cocaine, opiates, benzodiazepine, buprenorphine, methodone, synthetic marijuana, alcohol, oxycodone, tricylics anti depressant, phenocyclidine, barbiturates, propoyphene and adulterants.

Preferably, the health related information comprises one or more of the following tests:

Pulmonary Function Tests comprising one or more of: Obesity Parameters: BMI, BAI, SBMI, BFC, VFC, BA, BMR; Pulmonary Function Tests: FVC, MVV, SVC (Pre/Post); Other Parameters: SPO2, Hb, Heart Rate.

Liver Function Tests comprising one or more of: Obesity Parameters: BMI, BAI, SBMI BFC, VFC, BA, BMR; Liver Function Tests: Bilirubin, Urobilinogen;

Kidney Function Tests comprising one or more of: Obesity Parameters: BMI, BAI, SBMI, BFC, VFC, BA, BMR; Kidney Function Tests: Creatinine, Uric Acid Renal; Other Parameters: Creatinine, Miro Albumin, Protein Urine Parameters: Micro Albumin, Protein, Creatinine;

Infection Diseases comprising one or more of: Screening of Infectious Diseases: dengue, cholera, H1N1, Typhoid, Ebola, Chikungunya and Malaria;

Cardiovascular Tests comprising one or more of: Complete Cardiac Profile: ECG, Heart Rate, BP, hsCRP*, K+*; Obesity Parameters: BMI, BAI, SBMI, BFC, VFC, BA, BMR; Complete Diabetes Profile: FBS, GRBS, HbA1C; Complete Lipid Profile: TC, HDL, TG, LDL, TC/HDL, VLDL;

Diabetic Function Tests comprising one or more of: Obesity Parameters: BMI, BAI, SBMI, BFC, VFC, BA, BMR; Complete Diabetes Profile: FBS, GRBS, HbA1C; Other Parameters: Creatinine, Uric Acid; Complete Lipid Profile: TC, HDL, TG, LDL, TC/HDL, VLDL; and

Cancer Screening Tests comprising one or more of: Cancer Screening: quick, low interference and minimal pain; Breast, Cervical, Gastro intestinal, Colorectal, Oral, Prostate.

### BRIEF DESCRIPTION OF DRAWINGS

At least one example of the invention will be described with reference to the accompanying drawings, in which:

FIG. 1A illustrates an environment of computing devices to which embodiments of the present invention may be implemented;

FIG. 1B illustrates a block diagram to illustrate an interaction of various components involved to implement the present invention, in accordance with an embodiment of the present invention;

FIG. 1C illustrates a solution flow to implement the present invention, in accordance with an embodiment of the present invention;

FIG. 1D illustrates chronic disease focused, in accordance with an embodiment of the present invention;

FIG. 2 illustrates a computer implemented method for diagnostic assessments, in accordance with an embodiment of the present invention; and

FIG. 2A illustrates predictive and deductive analysis and display dashboard, in accordance with an embodiment of the present invention.

It should be noted that the same numeral represents the same or similar elements throughout the drawings.

### DESCRIPTION OF EMBODIMENTS

While the present invention is described herein by way of example using embodiments and illustrative drawings, those skilled in the art will recognise that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claims. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers, or steps. Likewise, the term “comprising” is considered synonymous with the terms “including” or “containing” for applicable legal purposes. Any discussion of documents, acts, materials, devices, articles, and the like are included in the specification solely for the purpose of providing a context for the present invention. It is not suggested or represented that any or all these matters form part of the prior art base or were common general knowledge in the field relevant to the present invention.

New technologies have an impact on health and medical services, from the range of digital health technologies to automated health and diagnostic services. They help improving the health system and affected patients together with the health workforce. Modem technologies offer a great opportunity to help our ageing societies through the use of modem technologies, like telemedicine services, for example.

The use of smartphones has continued to increase over the years, with the total number of smartphone users worldwide projected to exceed the 2.5 billion mark in 2019.

According to the 2016 Deloitte Mobile Consumer Survey, Australia's smartphone penetration level sits at 84%—the fourth largest market globally after Norway (91%), South Korea (89%), and the Netherlands (87%). This makes Australia one of the foremost adopters of such technology.

The inventors recognise that smartphones and their use offer a simple and convenient way for people around to the world to access, use and share information. Accordingly, the inventors sought to develop a portable diagnostic computer system with the intention of giving power to the people in the most remote areas of world to track their health. This system is set to offer tremendous support for medical care throughout world, including regions with poor infrastructure, while also seeking to lower costs of healthcare.

The present invention is applicable not only for patients, but also for doctors or health practitioners since it allows stakeholders to monitor the prognosis and diagnosis of patients in real-time. This allows the management of different types of patients, at the same time, in the same digital platform.

In this regard the invention below has been discussed with the help of figures for clarity. However, a skilled addressee would appreciate that the invention is not limited to particular types of implementations that have been discussed below.

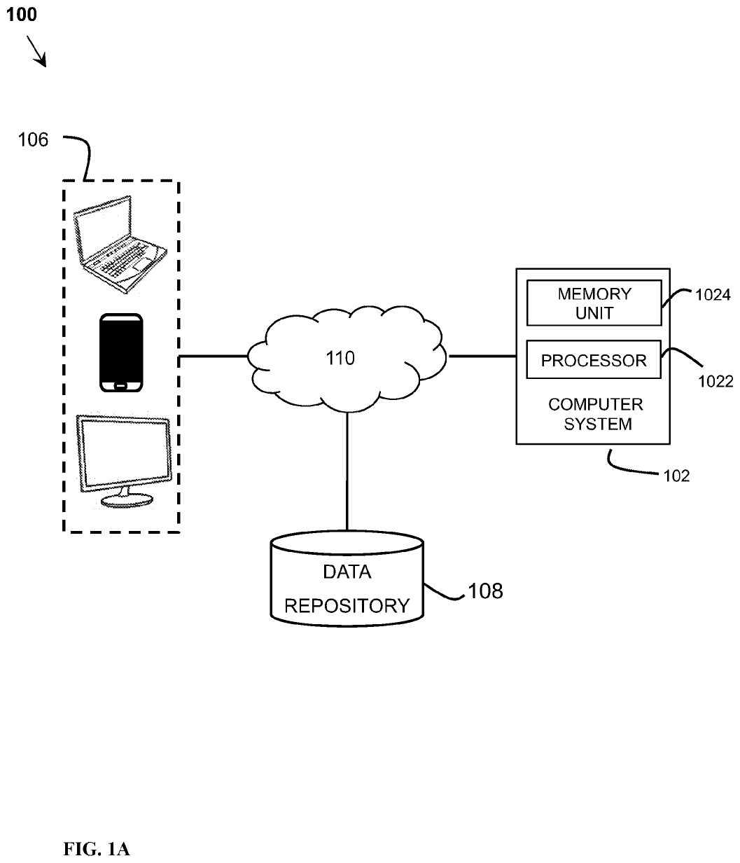

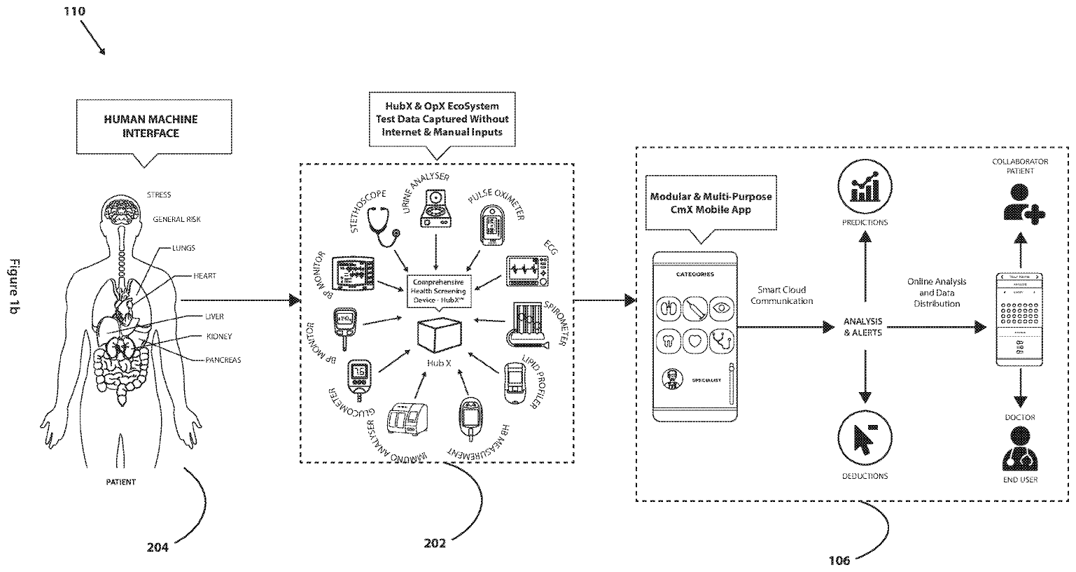

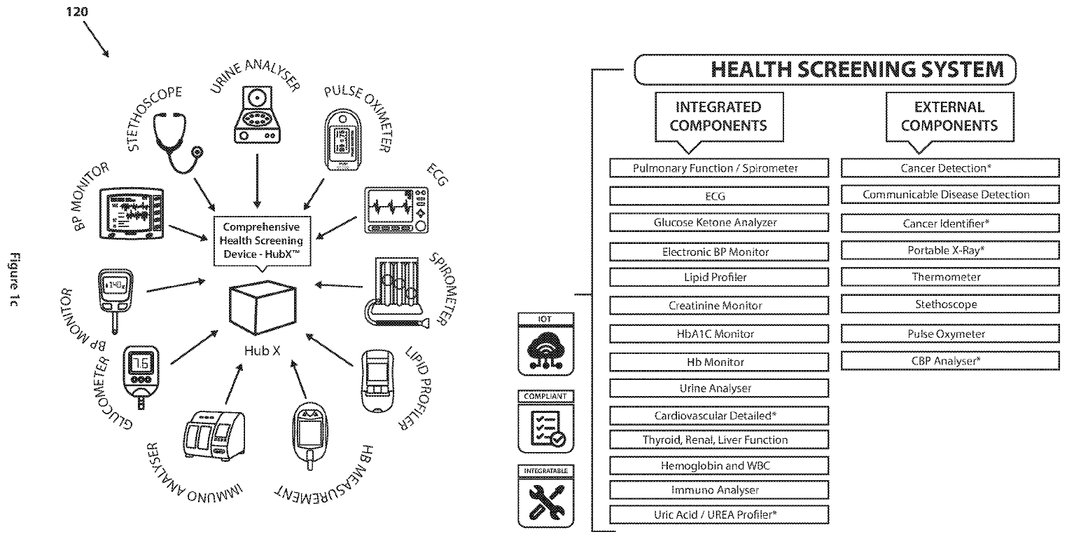

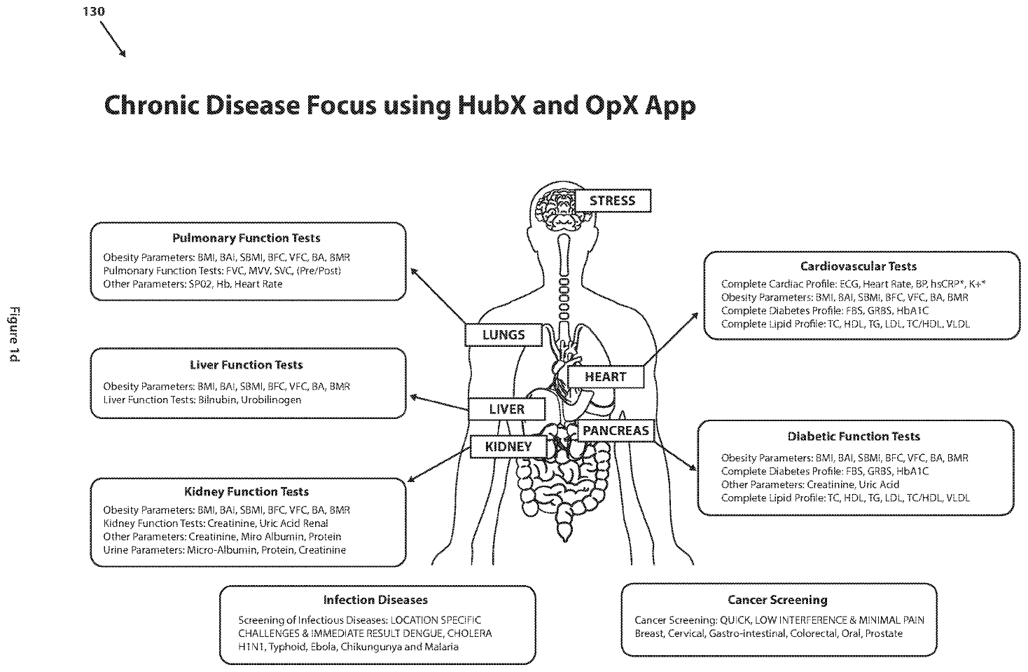

FIG. 1A illustrates an environment 100 of computing devices to which embodiments of the present invention may be implemented. As shown in FIG. 1A, the environment 100 comprises a computer system 102, connected one or more devices 106 associated with one or more patients, doctors, hospitals, healthcare organizations and other stake holders.

Herein, the one or more devices 106 enable the one or more patients, the doctors, the hospitals, the healthcare organizations, and other stake holders to access the present invention and accordingly, are selected from a laptop, a desktop PC, an AR/VR headset, a smartphone or a tablet a computer system 102 for providing an interactive experience.

Further, the computer system 102 is the primary component responsible for diagnostic assessments environment 100 talked about in the present invention. In that sense, the computer system 102 is envisaged to include computing capabilities such as a memory unit 1024 configured to store machine readable instructions. The machine-readable instructions may be loaded into the memory unit 1024 from a non-transitory machine-readable medium, such as, but not limited to, CD-ROMs, DVD-ROMs and Flash Drives. Alternately, the machine-readable instructions may be loaded in a form of a computer software program into the memory unit 1024. The memory unit 1024 in that manner may be selected from a group comprising EPROM, EEPROM and Flash memory. The computer system 102 also includes a processor 1022 operably connected with the memory unit 1024. In various embodiments, the processor 1022 may be a microprocessor 1022 selected from one of, but not limited to, an ARM based, or Intel based processor 1022 in the form of field-programmable gate array (FPGA), a general-purpose processor 1022 and an application specific integrated circuit (ASIC). Moreover, the computer system 102 may implement artificial intelligence and deep learning-based technologies for, but not limited to, data analysis, collating data and presentation of data in real-time.

In accordance with an embodiment of the present invention, a communication network 110 may also be used for connecting the components within the environment 100. The communication network 110 can be a short-range communication network 110 and/or a long-range communication network 110, wire or wireless communication network 110. The communication interface includes, but not limited to, a serial communication interface, a parallel communication interface or a combination thereof. The communication network 110 may be implemented using a number of protocols, such as but not limited to, TCP/IP, 3GPP, 3GPP2, LTE, IEEE 802.x etc. The communication network 110 may be wireless communication network 110 selected from one of, but not limited to, internet, for providing maximum coverage.

In addition, system may require a data repository 108 to implement the present invention. The data repository 108 shown in figure TA may be understood as a local storage (such as SSD, eMMC, Flash, SD card, etc) or a cloud-based storage. In any manner, the data repository 108 are envisaged to be capable of providing the data to the computer system 102, when the data is queried appropriately using applicable security and other data transfer protocols. The data repository 108 are further envisaged to store login data related to the user devices 106, system configurations, all accumulated health related data, history etc.

In one embodiment, as shown in FIG. 1B, the computer system 102 may contain one or more medical instruments 202 in communication with the processor 1022 and the one or more user devices. FIG. 1C illustrates a solution flow to implement the present invention, in accordance with an embodiment of the present invention. As shown in FIGS. 1B and 1C, the one or more medical instruments 202 may be, integrated instruments and/or external instruments such as, but not limited to, selected from a group comprising spirometer, electrocardiography sensor, glucose ketone analyser, electronic blood pressure (BP) monitor, Lipid Profiler, creatinine monitor, HbA1C monitor, Hb monitor, urine analyser, cardiovascular analyser, thyroid, renal, liver function analyser, haemoglobin and white blood cell (WBC) analyser, cancer screening, communicable disease detector, cancer identifier, portable X-Ray, uric acid/UREA profiler, Thermometer, stethoscope, pulse oximeter, CBP analyser, mammogram.

In one embodiment, the computer system 102 may comprise a plurality of wheels and back strap for quick and easy mobility. The plurality of wheels may facilitate the movement of the computer system 102 and the back strap may be used to strap the computer system 102 and carry the computer system 102 from one place to another.

In an exemplary embodiment of the present invention, the system may have components and specification as, but does not, by any mean, limit the scope of the present invention:
Weight: 14 kg
Structure Material: ABS Plastic
Dimensions: 400 mm(h)*350 mm*×400 mm (d)
Portability: Comes equipped with a customized protective bag, for easy transport in both urban and remote regions
Input Power: 230 250 V (AC), 5 Amp (Stored Power None Battery Operation 12 V (Inverted) (Fully Charged CAR battery can last approx. 10 days)
Wi Fi Requirement: Only for data retrieval and syncing offline data
Bluetooth Operation: Yes (printing)
Data Security: Encrypted offline storage, SSL transmission, Dual Authorization single authentication
Performance: Block chain cloud based architecture capable of holding and retrieving unlimited data
Health Insulation, Water and Dust Protection: RoHS Compliant, medical grade XL foam
Mobile Tablet: 10 inches (provided)
Operation: Using CmX and OpX App
Report Printing: Bluetooth thermal printer (provided)
Testing Protocols; Training and User Manuals: Written and online (provided)
Testing Modalities: Dry strip based reagents in a highly cost effective way Safe and easy to use with capillary blood and urine point of care testing modality Does not require use of a syringe or venepuncture for testing

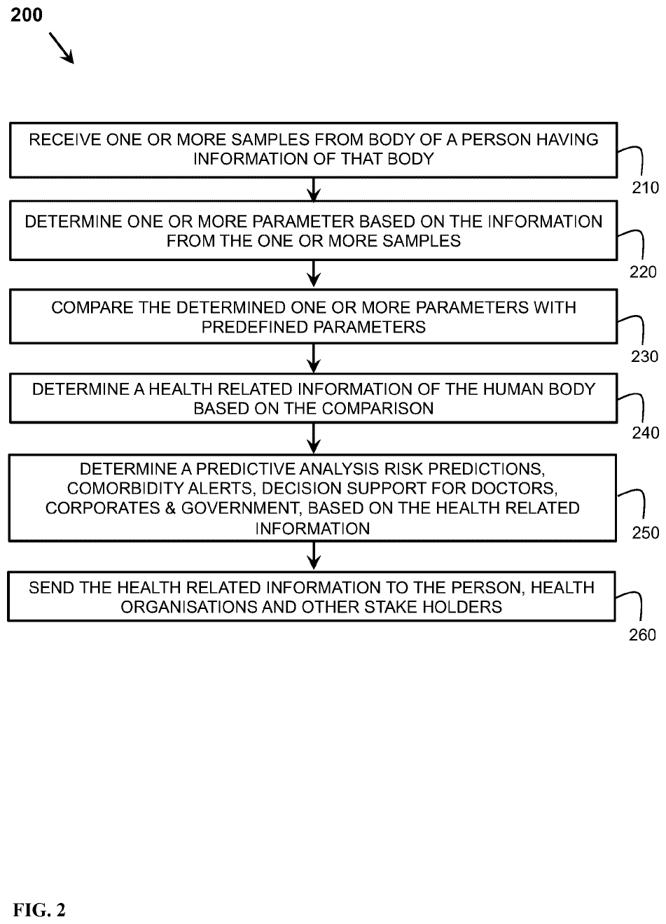

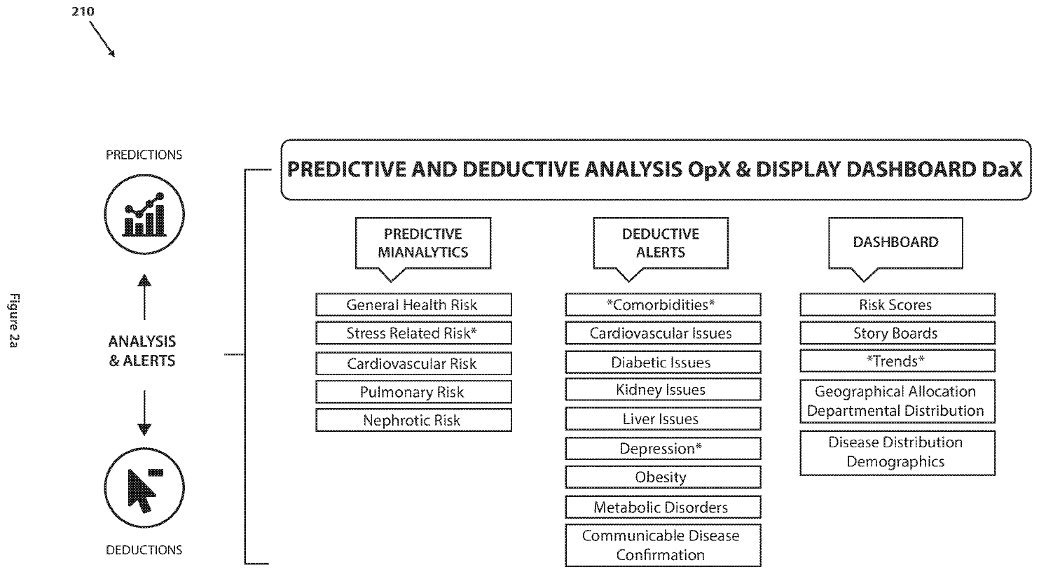

FIG. 2 illustrates a computer implemented method 200 for diagnostic assessments, in accordance with an embodiment of the present invention. Before explaining the method 200 of operation, it is important to understand that the present invention has broad scope and can have several applications. However, for the sake of better explanation, some examples may be used to explain the invention, but they should not be considered in a limiting sense.

As shown in FIG. 2, the method 200 begin at step 210 by receiving one or more samples from body of a person having information of that body.as shown in FIG. 2, any medical instrument 202 of the one or more medical instruments 202 configured to receive the one or more samples from the body. Herein the one or more samples are selected from a group comprising blood, tissue, saliva, or urine or a combination thereof. The computer system 102 uses capillary blood. However, by circumventing the use of venous blood and venous blood draw, the computer system 102 avoids the intervention of a medical doctor, in case there is none in close proximity.

Next, at step 220, the one or more medical instruments 202 may determine one or more parameter based on the information from the one or more samples. The one or more parameters such as, may be, but not limited to, temperature, blood pressure, Blood, Glucose, Protein, Ketone, pH, Nitrite, Leucocytes, Bilirubin, Urobilinogen, S.G., Colour, Clarity, clogging in veins and artilleries, heart beating rate, blood type, glucose, PH value, cardiovascular function, stress, liver function, prostate function, type of cells, growth of cells, Creatinine, Haemoglobin, Systolic and diastolic, Haematocrit, infection, influenza virus, bacteria etc.

Then, at step 230, the processor 1022 is configured to compare the determined one or more parameters with predefined parameters. The predefined parameters may be taken from a globally acceptable parameter defining the normal range or value of the determined one or more parameter. The comparison may be done using the predefined parameters of a globally acceptable healthy specimen.

Next, at step 240, the processor 1022 may be configured to determine health related information of the human body based on the comparison. FIG. 1D illustrates chronic disease focused, in accordance with an embodiment of the present invention. Herein the health related information the health related information is selected from a group comprising basic biometric data (including temperature), complete urine analysis, haemoglobin and white blood cell count, detailed cardiovascular function (electrocardiogram, heart rate), blood vitamin analysis, detailed diabetes blood glucose levels, renal function test, thyroid test, hypertension, liver function test, pulmonary function test, prostate function test, Cancer Markers, Tumour Markers, Infection Markers and many more like covid-19 antibody testing IgM, electrolyte measurement test (captured manually), and infectious disease tests (e.g., Malaria, Chikungunya, Typhoid, H1N1 etc.). The tests, symptoms, sample required in the test, parameters, and diagnostics have been illustrated in table 1, table 2 and table 3.

After that, at step 250, the processor 1022 is configured to determine a predictive analysis, risk predictions, comorbidity alerts, and decision support for doctors and/or corporates and/or government according to the requirements and needs of the test, based on the health-related information. FIG. 2A illustrates predictive and deductive analysis and display dashboard, in accordance with an embodiment of the present invention. As shown in FIG. 2A, in one embodiment, the risk predications may include, but not limited to, general health risk, stress related risk, cardiovascular risk, pulmonary risk, nephrotic risk. In one embodiment, the processor 1022 may provide deductive alerts such as, but not limited to, comorbidities, cardiovascular issues, diabetic issues, kidney issues, liver issues, depression, obesity, metabolic disorders, and communicable disease confirmation.

In accordance with an embodiment, the processor 1022 may further be configured to receive health information related to one or more patients for diagnostic assessments from health care practises such as doctors, hospitals, or health organizations. The health care practices may record the health-related information of the one or more patients and send the information to the computer system 102 for storing and processing and further, determine the predictive analysis risk predictions, comorbidity alerts, decision support for doctors, corporates and/or government, based on the health-related information.

Further, the processor 1022 may allow any patient of the one or more patients to view, share and maintain health data, preserving health records digitally and chronologically using the one or more devices 106 held by the patients. Furthermore, the processor 1022 may update information about any patient of the one or more patients thereby helping hospitals, medical stores and diagnostic centres.

In addition, the processor 1022 may facilitate one or more information dialogs between patients, doctors, hospitals, and all involved parties. The computer system 102 may be in communication with the patients, doctors, hospitals, and all involved parties using the communication network 110 may facilitate the dialogs.

Additionally, the processor 1022 may perform self-risk analysis tests for stress, and examine risk to develop certain chronic diseases. The processor 1022, in communication with the one or more medical instruments 202 may perform the self-risk analysis. Moreover, the processor 1022 may monitor prognosis and diagnosis of patients in real-time.

This is advantageous as to provide comprehensive health checks for workers in the workplace, remote communities/clinics or in the home. Further, the computer system 102 may facilitate monitoring and provide recommendation for prevention and management of health and stress related risks through an innovative and complete Electronic Health Record (I) integrated diagnostic tool. Further, the computer system 102 may help in refining processes in current care facilities, aid in diminishing budgets and reduce healthcare costs. The computer system 102 supports existing health care professionals to achieve mutual care delivery goals and conduct measurements of vital functions efficiently. Further, the computer system 102 may aid in decreasing the burden of chronic diseases for workers and their families. The computer system 102 may facilitate easy access to personal health record through the application. The computer system 102 may facilitate further access to primary care providers through Tele-Health and ePrescribing services. The computer system 102 may help in understanding the experiences of workers as consumers of health services and as primary industry employees. Further, the computer system 102 may track individual pathways through the health system for supported health monitoring and intervention. Further, the computer system 102 may help in:
Assist to monitor people who have been injured at work, are exposed to extreme work conditions i.e. underground surface, remote and hot conditions that are at risk of severe dehydration, have existing physical comorbidities, or are taking treatment for existing health needs;
Increase the efficiency of testing; the service delivery is ideal for time poor shift work;
Identify health trends and have a significant impact on improving existing barriers in the Australian Health system and target markets as a whole;
Higher compliance/adherence, better outcomes evidence and efficient data for payer formularies;
Improve organisation's competitive by attracting and retaining investors, clients and employees;
Demonstrates t‘e ′he’rt′ of a company; enhancing credibility, raise the ethical standard and contribution to the health of employees and the surrounding community. Thereby, creating a sense of employee satisfaction and loyalty;
Contribute to and support marketing tools: i.e. brand awareness;
Align company with industry charters: SRI (Social Responsibility Index) and GRI (Global Reporting Index);
Assist in stabilising the social and economic environment;
Generate new business ideas, and generate inquiries about business operations;
Enhance company's corporate image and ultimately return on investment.

The computer system 102 may be built on the strategic goals of the Australian Digital Health Strategy delivering a fully equipped service to improve health access and delivery, promote the collection of national data, reduce the cost of health care and decrease the burden of illness. Through the provision of the computer system 102, the aim is to:
Protect the health and safety of Indigenous Australians by adequately assess, predict, monitor, and treat the development of insidious, chronic and entirely preventable illnesses;
Provide efficiencies, improvements and solutions for the current health system by tracking health intervention experiences and individual pathways through the health system;
Improve the understanding of pathways through the health system, health outcomes, pathways and quality of care;
Create access to far richer national health data and allow detailed understanding of the use and outcomes of a vast range of digital health applications; and
Deliver a higher standard of clinical care and coordination using the latest innovative and cutting-edge technology.

Embodiments of the present invention provides a complete solution to create equal access to health care through the delivery of comprehensive health checks for Indigenous and rural communities, corporate industries, Primary industries, aging and disabled populations at clinics, remote communities/camps or in the comfort of their home. It provides onsite diagnostic support to current health care providers to improve medical supply, support better care for consumers, and support decision making by Primary care providers. The processes defined with the novel technology and near instant diagnostics promote efficient and effective health assessments and interventions, while reducing health costs. It helps consumers and their families decrease the health-related burden and improve treatment of chronic and high stress-related illnesses, through cost-effective service delivery initiatives. It educates and develops an awareness of illness to empower consumers to make healthy lifestyle choices for the prevention of chronic diseases and reach optimum health outcomes through the use of our cutting-edge technology and service delivery initiatives. No current technology allows for such a comprehensive range of testing with a device that has minimal bodily intrusion (5 drops of blood or urine). The devices of the preferred embodiment will provide for 150 plus tests within 15 minutes. The results are collated and will be provided electronically to health care professionals and the patient. The design of the equipment will allow portability and operability in all locations. No prior art provides the capability for a comprehensive full health check capturing mental and physical health check via blood, urine, electrocardiogram (ECG or EKG), blood pressure, spirometer, lipids, renal function, haemoglobin Hba1c, Glucose and urine analyser within 15 minutes that is mobile and portable.

It should be understood that the techniques of the present disclosure might be implemented using a variety of technologies. For example, the contactless payment described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data steams along a local network or a publicly accessible network such as the Internet.

It should also be understood that, unless specifically stated otherwise as apparent from the following discussion, it is appreciated that throughout the description, discussions utilizing terms such as “controlling” or “obtaining” or “computing” or “storing” or “receiving” or “determining” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

Table 1, Table 2 and Table 3 as detailed below provide a list of the tests that can be included in embodiments of the invention. Embodiments of the invention provide a comprehensive range of testing with a device that has minimal bodily intrusion (5 drops of blood or urine). The device provides for approximately 150 or more tests within 15 minutes. The results are collated and will be provided electronically to health care professionals and the patient. The design of the equipment allows portability and operability in all locations. It should be understood that the tests below are examples of tests only and other tests can be added as required according to the embodiment.

TABLE 1

| | Type of | Disease/ | Parameters | |
|---|---|---|---|---|
| S. No | test | Therapeutic area | captured | Notes |
| 1 | Urine | General Health, Liver | Blood, Bilirubin, | simple and quick |
| | Analysis | and Kidney disease, | Creatinine, | analysis based on |
| | | Diabetes and | Urobilinogen, | dip stick |
| | | Cardiovascular | Ketones, Protein, | methodology. |
| | | disease | Nitrites, | Multiple |
| | | | Microalbumin, | combination |
| | | | Glucose, pH, | parameters strips |
| | | | Leukocytes, | are available |
| | | | Ascorbic Acid, | |
| | | | Color, S.G., Clarity | |
| 2 | ECG | Cardiovascular | Various data points, | This is |
| | Analysis | disease | graphs and analysis | clinical/hospital |
| | | | | grade 12 lead ECG |
| 3 | Blood | Diabetes | Fasting and random | easy, accurate and |
| | Glucose and | | blood glucose, | with capillary blood |
| | Ketone | | Ketone | |
| | testing | | | |
| 4 | Blood | Cardiovascular | Systolic and | this is USFDA |
| | pressure | disease (CVD) | diastolic blood | approved |
| | | | pressure | component |
| 5 | Lipid | Cardiovascular | Complete Lipid | with <2 min turn- |
| | profiler | disease | Profile (Cholesterol, | around time |
| | (blood | | HDL, LDL, | |
| | sample) | | Triglyceride, | |
| | | | Cholesterol/HDL | |
| | | | Ratio, VLDL) | |
| 6 | Renal | Kidney Function | Blood creatinine, | very important |
| | function | and CVD | Uric Acid, Urea | parameter to |
| | | | | consider |
| | | | | comorbidity |
| | | | | conditions |
| 7 | HbA1C test | Diabetes | HbA1C % | this will validate the |
| | | | | diabetic condition |
| | | | | of the person |
| 8 | Hemoglobin | General Health | Hemoglobin value | |
| 9 | Spirometry | Pulmonary | Various data points, | can provide insights |
| | analysis | Function | graphs and analysis | into upper |
| | | | | respiratory track as |
| | | | | well as lower |
| | | | | respiratory track |
| | | | | disorders |
| 10 | Kit based | Infectious | Dengue, Malaria, | Specially for |
| | tests | diseases | Cholera, Typhoid, | remote areas and |
| | | | H1N1, Pneumonia | African countries |

TABLE 2

| | | | | |
|---|---|---|---|---|
| | | | Reaction | Measurement |
| Group | Item | Specimen | time | ranges |

Available Items

| Cardiac | CTN-I | WB/S/P | 15 Min | 0.1-50 | ng/mL |
|---|---|---|---|---|---|
| markers | CK-MB | WB/S/P | 15 Min | 0.3-100 | ng/mL |
| | D-Dimer | WB/P | 10 Min | 0.1-10 | mg/mL |
| | MYO | WB/S/P | 15 Min | 2-400 | ng/mL |
| | H-FABP | WB/S/P | 15 Min | 1-120 | ng/mL |
| | NT-ProBNP | | 15 Min | 18-35,000 | pg/mL |
| | Combo (CTN- | WB/S/P | 15 Min | Same as | |
| | I/CK-MB/MY0) | | | single item | |
| | Combo (CTN- | WB/S/P | 15 Min | Same as | |
| | I/CK-MB) | | | single item | |
| Infection | hsCRP/CRP | WB/S/P | 3 Min | 0.5-200 | mg/L |
| | PCT | WB/S/P | 10 Min | 0.1-50 | ng/mL |
| | SAA | WB/S/P | 10 Min | 5-100 | mg/L |
| | IL-6 | WB/S/P | 10 Min | 2-4,000 | pg/mL |
| Renal | CYS C | WB/S/P | 3 MIn | 0.5-10 | mg/L |
| Function | MAU | U | 3 MIn | 10-200 | mg/L |
| | BZ-MG | WB/S/P | 3 MIn | 0.5-20 | mg/L |
| | NGAL | WB/S/P | 3 MIn | 25-1,600 | ng/mL |
| Diabetes | HbA1c | WB | 5 Min | 4-16% | |
| Harmone | b-hCG | S/P | 15 Min | 5-10,000 | mlU/mL |
| | TSH | WB/S/P | 15 Min | 0.1-100 | mlU/mL |
| | T3 | S/P | 10 Min | 0.61-10.00 | nmol/L |
| | T4 | S/P | 15 Min | 20-310 | nmol/L |
| | LH | WB/S/P | 10 Min | 1-200 | mlU/mL |
| Drug of | MOR | H | 5 Min | 0.10-10.00 | ng/mg |
| abuse | MET | H | 5 Min | 0.10-10.00 | ng/mg |
| | KET | H | 5 Min | 0.10-10.00 | ng/mg |
| | Combo | H | 5 Min | Same as | |
| | (MOR/MET/KET) | | | single item | |
| Others | FER | S/P | 10 Min | 5-1,500 | ng/mL |

Upcoming Items

| | | | | | |
|---|---|---|---|---|---|
| Tumor | PSA | S/P | 10 Min | 2-100 | ng/mL |
| markers | AFP | S/P | 10 Min | 5-400 | ng/mL |
| | CEA | S/P | 10 Min | 1-500 | ng/mL |
| | IFOB | F | 10 Min | 25-1,000 | ng/mL |
| Diabetes | C-P | WB/S/P | 10 Min | 0.4-20 | ng/mL |
| | INS | WB/S/P | 10 Min | 0.5-100 | mlU/L |
| Drug of | THC | H | 5 Min | 0.02-2.0 | ng/mg |
| abuse | COC | H | 5 Min | 0.20-10.00 | ng |

WB = Whole blood; S = Serum; P = Plasma; F = Feces; U = Urine; min = minutes; H = Hair

TABLE 3
TEST LIST

| | | | Sample | Sample | Test | Reference | | Linearity | | |
|---|---|---|---|---|---|---|---|---|---|---|
| CAT | Parameters | Principle | type | size | time | range | | range | | Clinical application |
| Infection | High sensitivity | Nephelometry | whole | 2.5 μl | 90 S | <2 | mg/L | 0.5~200 | mg/L | Discriminate bacteria |
| | C reaction | | blood | | | | | | | and virus infection, |
| | protein(CRP) | | | | | | | | | especially for infant |
| | Full range CRP | Nephelometry | whole | 2.5 μl | 90 S | <10 | mg/L | 0.5~200 | mg/L | Discriminate bacteria |
| | | | blood | | | | | | | and virus infection |
| | Procalcitonin(PCT) | Nephelometry | Serum | 20 μl | 480 S | <0.5 | ng/mL | 0.2~60 | ng/mL | Sepsis, antibiotics |
| | | | | | | | | | | monitoring |
| | Serum amyloid A | Nephelometry | whole | 2.5 μl | 130 S | <6.4 | mg/L | 3~200 | mg/L | Discriminate bacteria |
| | (SAA) | | blood | | | | | | | infection with PCT, |
| | | | Serum | | | | | | | CRP for infant |
| Cardio- | (H-FABP) | Nephelometry | Serum | 20 μl | 360 S | <6.2 | ng/mL | 2.5~160 | ng/mL | The most sensitive |
| vascular | Human-fatty acid | | | | | | | | | indicator for early |
| | Binding protein | | | | | | | | | detection of AMI, can |
| | | | | | | | | | | be detected in 30 |
| | | | | | | | | | | minutes after |
| | | | | | | | | | | Myocardial damage |
| | Myohemoglobin | Nephelometry | Serum | 10 μl | 300 S | <90 | μg/L | 3~500 | μg/L | Early indicator of AMI, |
| | (Myo) | | | | | | | | | can be detected in 1 hour |
| | | | | | | | | | | after Myocardial |
| | | | | | | | | | | damage |
| | Cardiac Tropnin I | Nephelometry | Serum | 20 μl | 330 S | <1.68 | ng/mL | 0.05~30 | ng/mL | The most sensitive and |
| | (cTnI) | | | | | | | | | most specific marker of |
| | | | | | | | | | | myocardial cell death, |
| | | | | | | | | | | becoming the “gold |
| | | | | | | | | | | standard” for the |
| | | | | | | | | | | diagnosis of myocardial |
| | | | | | | | | | | infarction. |
| | Lipoprotein (a) | Nephelometry | Serum | 10 μl | 330 S | ≤300 | mg/L | 10~1000.0 | | Predictive factors of |
| | (LP(a)) | | | | | | | mg/L | | Coronary heart disease, |
| | | | | | | | | | | atherosclerosis, |
| | | | | | | | | | | cerebral infarction |
| | D-Dimer | Nephelometry | Plasma | 15 μl | 180 S | <1.35 | mg/L | 0.5~20 | mg/L | Early diagnotic for |
| | | | | | | | | | | DIC, DVT screening, PE |
| | | | | | | | | | | diagnostic, thrombolytic |
| | | | | | | | | | | therapy monitoring |
| | Lipoprotein | Nephelometry | Serum | 15 μl | 300 S | <175 | ng/mL | 0.5~800 | ng/mL | High specificity |
| | phospholipase A2 | | | | | | | | | markers of Vascular |
| | (Lp-PLA2) | | | | | | | | | inflammation, |
| | | | | | | | | | | dynamic monitoring |
| | | | | | | | | | | A2 for risk and efficacy |
| | | | | | | | | | | evaluation on variety of |
| | | | | | | | | | | atherosclerosis-related |
| | | | | | | | | | | cardiovascular |
| | | | | | | | | | | and cerebrovascular |
| | | | | | | | | | | embolism disease |
| Renal | Hemoglobin A1c | Nephelometry | whole | 10 μl | 300 S | 3.8-5.8% | | 2~14% | | Gold standard for |
| Function | (HbA1c) | | blood | | | | | | | detecting diabetes |
| | Microalbumin | Nephelometry | Urine | 10 μl | 180 S | <25 | mg/L | 10~220 | mg/L | Early diagnosis of |
| | (mAlb) | | | | | | | | | kidney injury |
| | Cystine (CysC) | Nephelometry | Serum | 5 μl | 300 S | <1.16 | mg/L | 0.2~9 | mg/L | Early and effective |
| | | | | | | | | | | indicator of renal |
| | | | | | | | | | | dysfuction and acute |
| | | | | | | | | | | renal failure. |
| | Neutrophil | Nephelometry | Serum | 5 μl | 330 S | <180 | ng/mL | 10~5000 | ng/mL | Early and sensitive |
| | Gelatinase | | | | | | | | | biomarker of renal |
| | Associated | | | | | | | | | injury, the NGAL |
| | Lipocalin (NGAL) | | | | | | | | | content increased |
| | | | | | | | | | | within 2 hours after |
| | | | | | | | | | | renal injury. |
| | α1-microglobulin | Nephelometry | Urine | 5 μl | 360 S | <12 | mg/L | 5~90 | mg/L | Sensitive indicator of |
| | (α1-MG) | | | | | | | | | tubular resorption |
| | | | | | | | | | | imparired |
| | β2-microglobulin | Nephelometry | Serum | 5 μl | 360 S | 1.09-2.53 | mg/L | 0.2~20 | mg/L | A sensitive and specific |
| | (β2-MG) | | | | | | | | | diagnostic index of renal |
| | | | | | | | | | | tubular damage, and |
| | | | | | | | | | | sensitive indicator of |
| | | | | | | | | | | hypertensive and |
| | | | | | | | | | | diabetic renal |
| | | | | | | | | | | function damage |
| | Retinol binding | Nephelometry | Serum | 5 μl | 360 S | 30-60 | mg/L | 5~140 | mg/L | Early and Sensitive |
| | protein (RBP) | | | | | | | | | indicator of renal |
| | | | | | | | | | | function impaired, a |
| | | | | | | | | | | specific indicator of |
| | | | | | | | | | | early clinical evaluation |
| | | | | | | | | | | of nutritional status. |
| Rheumatism | Antistreptolysin O | Nephelometry | Serum | 2.5 μl | 180 S | <200 | lU/mL | 30~600 | lU/mL | Diagnosis of hemolytic |
| | (ASO) | | | | | | | | | streptococcus infection, |
| | | | | | | | | | | specific indicator of |
| | | | | | | | | | | active rheumatic fever |
| | Rheumatoid factor | Nephelometry | Serum | 5 μl | 180 S | <30 | lU/mL | 5~160 | lU/mL | The most widely used |
| | (RF) | | | | | | | | | clinical test indicator for |
| | | | | | | | | | | rheumatoid arthritis |
| | | | | | | | | | | (RA) diagnosis and |
| | | | | | | | | | | treatment. High |
| | | | | | | | | | | sensitivity, The highest |
| | | | | | | | | | | positive rate with lower |
| | | | | | | | | | | specificity. |
| | Anti-CCP | Nephelometry | Serum | 10 μl | 180 S | <45 | U/mL | 5~100 | U/mL | High sensitivity and |
| | | | | | | | | | | specificity of early |
| | | | | | | | | | | diagnostic for RA, the |
| | | | | | | | | | | sensitive indicator of |
| | | | | | | | | | | identify aggressive, |
| | | | | | | | | | | noninvasive RA. |
| Gastrict | PepsinogenI(PGI) | Nephelometry | Serum | 15 μl | 300 S | >70 | ng/mL | 2.5~160 | ng/mL | Indicate the functional |
| function | | | | | | | | | | status of the gastric |
| | | | | | | | | | | fundus and gastric |
| | | | | | | | | | | mucosa |
| | PepsinogenII(PGII) | Nephelometry | Serum | 15 μl | 300 S | <27 | ng/mL | 2.0~80 | ng/mL | Indicate the functional |
| | | | | | | | | | | status of the Antral |
| | | | | | | | | | | mucosa |
| Liver | Prealbumin (PA) | Nephelometry | Serum | 5 μl | 300 S | 200-400 | mg/L | 10~800 | mg/L | Diagnosis of |
| function | | | | | | | | | | malnutrition, indicators |
| | | | | | | | | | | of liver dysfunction |
| | Transferrin(TRF) | Nephelometry | Serum | 5 μl | 300 S | 2.0-3.6 | g/L | 0.2~7.4 | g/L | Diagnosis of iron |
| | | | | | | | | | | deficiency and liver |
| | | | | | | | | | | disease |
| Auto- | Immune globulin | Nephelometry | Serum | 2.5 μl | 300 S | 0.7-4.0 | g/L | 0.5~6.0 | g/L | The normal IgA |
| immunity | A (IgA) | | | | | | | | | concentration is 0.7-4.0 |
| | | | | | | | | | | g/L, if increased or |
| | | | | | | | | | | decreased indicating |
| | | | | | | | | | | have got autoimmune |
| | | | | | | | | | | disease. 1, IgA |
| | | | | | | | | | | increased: >4.0 g/L, |
| | | | | | | | | | | indicating have got |
| | | | | | | | | | | connective tissue |
| | | | | | | | | | | disease, liver disease, |
| | | | | | | | | | | infectious diseases 2, |
| | | | | | | | | | | IgA decreased: <0.7 g/L, |
| | | | | | | | | | | common in Autoimmune |
| | | | | | | | | | | diseases, transfusion |
| | | | | | | | | | | reactions, primary |
| | | | | | | | | | | non-gamma |
| | | | | | | | | | | globulinemia, secondary |
| | | | | | | | | | | immunodeficiency and |
| | | | | | | | | | | malabsorption syndrome |
| | Immune globulin | Nephelometry | Serum | 10 μl | 300 S | 0.4-2.3 | g/L | 0.25~5.00 | g/L | The normal IgM |
| | M (IgM) | | | | | | | | | concertration is 0.4-2.3 |
| | | | | | | | | | | g/L, if increased or |
| | | | | | | | | | | decreased indicating |
| | | | | | | | | | | have got autoimmune |
| | | | | | | | | | | disease. 1, IgM |
| | | | | | | | | | | increased: >2.3 g/L, |
| | | | | | | | | | | indicating have got |
| | | | | | | | | | | connective tissue |
| | | | | | | | | | | disease, liver disease, |
| | | | | | | | | | | infectious diseases 2, |
| | | | | | | | | | | IgM decreased: <0.4 g/L, |
| | | | | | | | | | | indicating have got |
| | | | | | | | | | | Hodgkin's disease, |
| | | | | | | | | | | leukemia |
| | Immune globulin | Nephelometry | Serum | 20 μl | 300 S | ≤100 | IU/ml | 50.0 IU/mL~960.0 | | It is mainly used for the |
| | E (IgE) | | | | | | | IU/mL | | evaluation of immune |
| | | | | | | | | | | function and auxiliary |
| | | | | | | | | | | diagnosis of immune |
| | | | | | | | | | | diseases. |
| | Immune globulin | Nephelometry | Serum | 2.5 μl | 300 S | 7-16 | g/L | 2.0~40.0 | g/L | The normal IgG |
| | G (IgG) | | | | | | | | | concertration is 7-16 g/L, |
| | | | | | | | | | | if increased or decreased |
| | | | | | | | | | | indicating have got |
| | | | | | | | | | | autoimmune disease. 1, |
| | | | | | | | | | | IgG increased: >16 g/L, |
| | | | | | | | | | | indicating have got |
| | | | | | | | | | | Hodgkin's disease, |
| | | | | | | | | | | leukemia 2, IgG |
| | | | | | | | | | | decreased: <7 g/L, |
| | | | | | | | | | | common in Various |
| | | | | | | | | | | congenital or acquired |
| | | | | | | | | | | humoral immune |
| | | | | | | | | | | deficiency disorders, |
| | | | | | | | | | | combined |
| | | | | | | | | | | immunodeficiency |
| | | | | | | | | | | disease |
| Tumor | Alpha fetoprotein | Nephelometry | Serum | 20 μl | 300 S | ≤20 | ng/Ml | 5-800 | ng/mL | Determine the content |
| Marker | (AFP) | | | | | | | | | of alpha fetoprotein |
| | | | | | | | | | | (AFP) in human serum |
| | | | | | | | | | | to aid in diagnosis |
| | | | | | | | | | | of primary hepatic |
| | | | | | | | | | | carcinoma. |

1. A computer system for diagnostic assessments, the computer system comprising:
one or more medical instrument sensors, wherein any medical instrument of the one or more medical instruments configured to:
receive one or more samples from body of a person having information of that body;
determine one or more parameter based on the information from the one or more samples;
a memory unit configured to store machine-readable instructions; and
a processor operably connected with the memory unit, the processor obtaining the machine-readable instructions from the memory unit, and being configured by the machine-readable instructions to:
compare the determined one or more parameters with predefined parameters;
determine a health related information of the human body based on the comparison; and
determine a predictive analysis risk predictions, comorbidity alerts, decision support for doctors, corporates and government, based on the health related information; and
a communication network configured to:
send the health related information to one or more user devices held by the person, health organisations and other stake holders.

2. The computer system as claimed in claim 1, wherein the one or more medical instruments may be selected from a group comprising spirometer, electrocardiography sensor, glucose ketone analyser, electronic blood pressure (BP) monitor, Lipid Profiler, creatinine monitor, HbA1C monitor, Hb monitor, urine analyser, cardiovascular analyser, thyroid, renal, liver function analyser, haemoglobin and white blood cell (WBC) analyser, cancer screening, communicable disease detector, cancer identifier, portable X-Ray, uric acid/UREA profiler, Thermometer, stethoscope, pulse oximeter, CBP analyser, mammogram.

3. The computer system as claimed in claim 1, wherein the one or more samples are selected from a group comprising blood, tissue, saliva, or urine or a combination thereof.

4. The computer system as claimed in claim 1, comprising a plurality of wheels and back strap for quick and easy mobility.

5. The computer system as claimed in claim 1, wherein the health related information is selected from a group comprising basic biometric data (including temperature), complete urine analysis, haemoglobin and white blood cell count, detailed cardiovascular function, detailed diabetes blood glucose levels, renal function test, thyroid test, hypertension, liver function test, pulmonary function test, prostate function test, Cancer Markers, Tumour Markers, Infection Markers, electrolyte measurement test, and infectious disease tests.

6. The computer system as claimed in claim 1, wherein the communication network is wireless and internet-enabled with an integrated 4G dongle.

7. The computer system as claimed in claim 1, wherein the processor is further configured to:
receive health information related to one or more patients for diagnostic assessments from doctors, hospitals or health organizations;
allow any patient of the one or more patients to view, share and maintain health data, preserving health records digitally and chronologically;
update information about any patient of the one or more patients thereby helping hospitals, medical stores and diagnostic centres;
facilitate one or more information dialogs between patients, doctors, hospitals and all involved parties;
perform self-risk assessment tests for stress, and also examine risk to develop certain chronic diseases; and
monitor prognosis and diagnosis of patients in real-time.

8. The computer system as claimed in claim 1, wherein the one or more user devices are selected from, but not limited to, a laptop, a desktop PC, an AR/VR headset, a smartphone or a tablet.

9. A computer implemented method for diagnostic assessments, wherein the method comprises steps of:
receiving one or more samples from body of a person having information of that body;
determining one or more parameter based on the information from the one or more samples;
comparing the determined one or more parameters with predefined parameters;
determining a health related information of the human body based on the comparison; and
determining a predictive analysis risk predictions, comorbidity alerts, decision support for doctors, corporates and government, based on the health related information; and
sending the health related information to the person, health organisations and other stake holders.

10. The computer implemented method as claimed in claim 8, further comprising steps of:
receiving health information related to one or more patients for diagnostic assessments from doctors, hospitals or health organizations;
allowing any patient of the one or more patients to view, share and maintain health data, preserving health records digitally and chronologically;
updating information about any patient of the one or more patients thereby helping hospitals, medical stores and diagnostic centres;
facilitating one or more information dialogs between patients, doctors, hospitals and all involved parties;
performing self-risk assessment tests for stress, and also examine risk to develop certain chronic diseases; and
monitoring prognosis and diagnosis of patients in real-time.

11. A portable medical device for diagnostic assessments, wherein the portable medical device comprises:
a plurality of medical instruments each medical instrument including one or more sensors, wherein each sensor of the one or more medical instruments is configured to receive one or more samples from body of a person having information of that body;
a processor to determine a plurality of parameters based on the information from the one or more samples;
a memory unit operably connected to the processor and configured to store machine-readable instructions, the processor obtaining the machine-readable instructions from the memory unit, and being configured by the machine-readable instructions to compare the determined the plurality of parameters with predefined parameters to thereby determine health related information of the human body based on the comparison.

12. A portable medical device for diagnostic assessments according to claim 11 wherein the determined health related information of the human body comprises one or more of:
a predictive analysis risk predictions;
comorbidity alerts; and
information for healthcare professionals, based on the health related information.

13. A portable medical device for diagnostic assessments according to claim 12 including a communication network configured to send the health related information to one or more user devices.

14. A portable medical device for diagnostic assessments according to claim 13 wherein the one or more samples from body of a person having information of that body comprise one or more of the following: blood; tissue; saliva; urine; or faeces.

15. A portable medical device for diagnostic assessments according to claim 14 wherein the user devices comprise one or more of the following: a laptop computer; a desktop computer; a portable electronic device such as a mobile phone or tablet; a patient database.

16. A portable medical device for diagnostic assessments according to claim 14 wherein determined health related information of the human body is determined within about 15 minutes.

17. A portable medical device for diagnostic assessments according to claim 16 wherein the predictive analysis risk predictions include predictions of one: diabetes; health comorbidities; cardiovascular disorders; stress related illness; kidney disease; respiratorydisorders; liver disease; cancer.

18. A portable medical device for diagnostic assessments according to claim 17 wherein the plurality of medical instruments includes one or more of the following integrated components:
Pulmonary Function/Spirometer; ECG; Glucose Ketone Analyzer; Electronic BP Monitor; Lipid Profiler; Creatinine Monitor; HbA1C Monitor; Hb Monitor; Urine Analyser; Cardiovascular Monitor; Thyroid, Renal, Liver Function monitor; Hemoglobin and WBC; Immuno Anaylser; Uric Acid/UREA Profiler.

19. A portable medical device for diagnostic assessments according to claim 18 wherein the plurality of medical instruments includes one or more of the following external components: Cancer Detection; Communicable Disease Detection; Cancer Identifier; Portable X-Ray; Thermometer; Stethoscope; Pulse Oxymeter; CBP Analyser.

20. A portable medical device for diagnostic assessments according to claim 19 wherein the health related information comprises one or more of the following tests:
Pulmonary Function Tests comprising one or more of: Obesity Parameters: BMI, BAI, SBMI, BFC, VFC, BA, BMR; Pulmonary Function Tests: FVC, MVV, SVC (Pre/Post); Other Parameters: SPO2, Hb, Heart Rate;
Liver Function Tests comprising one or more of: Obesity Parameters: BMI, BAI, SBMI BFC, VFC, BA, BMR; Liver Function Tests: Bilirubin, Urobilinogen;
Kidney Function Tests comprising one or more of: Obesity Parameters: BMI, BAI, SBMI, BFC, VFC, BA, BMR; Kidney Function Tests: Creatinine, Uric Acid Renal; Other Parameters: Creatinine, Miro Albumin, Protein Urine Parameters: Micro Albumin, Protein, Creatinine;
Infection Diseases comprising one or more of: Screening of Infectious Diseases: dengue, cholera, H1N1, Typhoid, Ebola, Chikungunya and Malaria;
Cardiovascular Tests comprising one or more of: Complete Cardiac Profile: ECG, Heart Rate, BP, hsCRP*, K+*; Obesity Parameters: BMI, BAI, SBMI, BFC, VFC, BA, BMR; Complete Diabetes Profile: FBS, GRBS, HbA1C; Complete Lipid Profile: TC, HDL, TG, LDL, TC/HDL, VLDL;
Diabetic Function Tests comprising one or more of: Obesity Parameters: BMI, BAI, SBMI, BFC, VFC, BA, BMR; Complete Diabetes Profile: FBS, GRBS, HbA1C; Other Parameters: Creatinine, Uric Acid; Complete Lipid Profile: TC, HDL, TG, LDL, TC/HDL, VLDL; and
Cancer Screening Tests comprising one or more of: Cancer Screening: quick, low interference and minimal pain; Breast, Cervical, Gastro intestinal, Colorectal, Oral, Prostate.
